(19) United States
(12) Patent Application Publication
(10) Pub. No.: US 2003/0097670 A1
(43) Pub. Date: May 22, 2003

(54) Expression of polypeptides in rod outer segment membranes

(76) Inventors: Krzysztof Palczewski, Bellevue, WA (US); Ning Li, San Diego, CA (US); Juan Ballesteros, Encinitas, CA (US)

Correspondence Address: CATHRYN CAMPBELL, CAMPBELL & FLORES LLP, 4370 LA JOLLA VILLAGE DRIVE, 7TH FLOOR, SAN DIEGO, CA, 92122, US

(21) Appl. No.: 09/990,185

(22) Filed: Nov. 21, 2001

(51) Int. Cl. A01K 67/027; C12P 21/02; C12N 5/06; C07K 14/705

(52) U.S. Cl. 800/18; 435/354; 435/320.1; 530/350

(57) ABSTRACT

The invention provides a gene targeting construct containing a transgene encoding a polypeptide that contains a rod outer segment (ROS) targeting signal. The transgene is flanked by 5′ and 3′ DNA sequences that are homologous to the rhodopsin gene. Homologous recombination between the construct and a rhodopsin gene results in operable association between the transgene and a rod-specific regulatory sequence. The invention also provides cells and animals whose genome contain a functional disruption of one or both endogenous rhodopsin gene alleles, and a transgene encoding a polypeptide that contains a ROS targeting signal operably associated with a rod-specific regulatory sequence. The invention constructs, cell and animals can be used to isolate transgenic polypeptides from the ROS membrane.

### BACKGROUND OF THE INVENTION

### FIELD OF THE INVENTION

The invention relates generally to the fields of protein structural biology and pharmaceutical design and, more specifically, to DNA constructs, cells and animals suitable for producing and isolating homogeneous proteins.

### BACKGROUND INFORMATION

Membrane proteins are critical for cellular communication, electrical and ion balance, structural integrity of cells, cell adhesion, and other functions. Among membrane proteins, G-protein coupled receptors (GPCRs) are of particular interest, because they form one of the largest and most diverse groups of receptor proteins. The more than 400 nonosensory GPCRs in the human genome are involved in the regulation of a multitude of physiological process. Several hundred other GPCRs are involved in sensing light, odor and taste. More than 40% of the total sales of available drugs are aimed at GPCRs, and GPCRs are being actively investigated throughout the pharmaceutical industry.

Structural models of proteins have proven useful in predicting mechanisms of ligand binding, predicting the effect of disease-causing mutations, and supporting drug design. However, obtaining atomic resolution structures of membrane proteins has proven technically challenging, in large part because expression of membrane proteins in tissue culture systems, which has conventionally been used to obtain the desired protein in large amounts, yields proteins that lack certain of the post-translational modifications found in native proteins, such as fatty acylation, phosphorylation and N- and O-linked glycosylation, or that have altered patterns of such modifications compared to native proteins. These differences can affect the stability of the protein, making it hard to isolate in soluble form. Additionally, the exact post-translational modifications differ from molecule to molecule in tissue culture systems. This heterogeneity detrimentally affects the ability to form suitable crystals for structural studies. For example, the GPCR bovine rhodopsin, purified either from recombinant mammalian cell lines or baculovirus/insect cells, exhibits differences in the amount of N-glycosylation as compared to rhodopsin isolated from bovine rod cells, and also exhibits a more diffuse band on an electrophoresis gel, indicative of heterogeneity (Reeves et al., Proc. Natl. Acad. Sci. USA 93:11487-11492 (1996)).

To date, only a single GPCR crystal structure has been determined, that of bovine rhodopsin (Palczewski et al., Science 289:739-745 (2000)). Rhodopsin is a GPCR involved in the transmission of light signals in the retina. To prepare high quality crystals, rhodopsin was isolated from the membranes of the rod outer segment of bovine retinas, where it constitutes about 90% of the total protein content.

Unfortunately, natural sources of most other membrane proteins in similar abundance and purity are not available. Additionally, as described above, proteins isolated from recombinant sources in tissue culture are generally heterogeneous and thus have not proven suitable for structural studies. Thus, there exists a need for a method for producing proteins, and particularly membrane proteins such as GPCRs, in high abundance, purity and homogeneity. Such proteins can be used for structural studies as well as for other research and therapeutic applications. The present invention satisfies this need and provides related advantages as well.

### SUMMARY OF THE INVENTION

The invention provides a gene targeting construct. The construct contains a transgene encoding a polypeptide that contains a rod outer segment (ROS) targeting signal. The transgene is flanked by 5′ and 3′ DNA sequences which are homologous to the mouse rhodopsin gene. Homologous recombination between the construct and a mouse rhodopsin gene results in operable association between the transgene and a rod-specific regulatory sequence.

The invention also provides a mouse cell whose genome contains a functional disruption of one or both endogenous rhodopsin gene alleles, and a transgene encoding a polypeptide that contains a ROS targeting signal operably associated with a rod-specific regulatory sequence.

Further provided is a mouse whose genome contains a functional disruption of one or both endogenous rhodopsin gene alleles, and a transgene encoding a polypeptide that contains a ROS targeting signal operably associated with a rod-specific regulatory sequence.

### BRIEF DESCRIPTION OF THE DRAWINGS

FIG. 1 shows an exemplary gene targeting construct and targeting strategy for expressing a transgenic polypeptide in the rod outer segment (ROS) membrane of mouse retina. The genomic clone contains five exons (E1 to E5) of the mouse rhodopsin gene (also known as the opsin gene or rod opsin gene). The transgene is a G-protein coupled receptor (GPCR) tagged on its C-terminus with a ROS targeting signal (hatched box). Expression of the transgene is under the control of the mouse rhodopsin promoter (5′ of the arrow). An excisable positive selection marker (neo flanked by loxP sites) and a negative selection marker (DTα) are indicated.

FIG. 2 shows a schematic diagram of an exemplary construct for expressing the human cannabinoid receptor 2 (CB2) in the rod outer segment of transgenic Xenopus laevis.

### DETAILED DESCRIPTION OF THE INVENTION

The invention provides animals that express transgenic polypeptides in the outer segment membrane of rod cells, as well as cells and constructs suitable for preparing such animals. The invention constructs can advantageously be designed so that homozygous animals produce little or no endogeous rhodopsin in the rod cells. The trangenic polypeptides expressed in the rod outer segment (ROS) membranes thus comprise a large percentage of the total ROS membrane protein content, and can be readily purified in large amounts. The transgenic polypeptides are also substantially homogenous in their post-translational modifications. Therefore, polypeptides produced by the invention animals and methods are useful for structural studies to elucidate their molecular mechanisms and ligand interactions, thereby providing useful information for drug design. The ROS membrane-expressed proteins are also useful in other applications known in the art for which high quality protein preparations are required or advantageous, including functional studies; screening for ligands, agonists and antagonists; preparation of antibodies; and preparation of pharmaceuticals.

In one embodiment, the invention provides a gene targeting construct that contains a transgene encoding a polypeptide comprising a rod outer segment (ROS) targeting signal. The transgene is flanked by 5′ and 3′ DNA sequences which are homologous to a rhodopsin (also known as opsin or rod opsin) gene. Following homologous recombination between the construct and a rhodopsin allele, the transgene and a rod-specific regulatory sequence are operably associated and the rhodopsin allele is functionally disrupted. An invention gene targeting construct can advantageously be used, for example, to prepare animals that express the polypeptide encoded by the transgene in the rod outer segment membrane, and to prepare suitable ES cells for use in making such animals.

As used herein, the term “transgene” refers to a DNA sequence which does not naturally occur at the rhodopsin gene locus. A transgene can encode any polypeptide for which expression in the rod outer segment membrane is desirable and for which an encoding sequence is known or can be determined. A large number of nucleotide sequences that encode human and non-human polypeptides are known in the art (see, for example, GenBank and other sequence databases), and others can be readily determined. Suitable coding portions, together with untranslated sequences important for mRNA stability and translation, can be sythesized or cloned by standard recombinant molecular biology methods (see, for example, Sambrook et al., Molecular Cloning: A Laboratory Manual, 3rd ed., Cold Spring Harbor Press, Plainview, N.Y. (2001); Ausubel et al. Current Protocols in Molecular Biology, John Wiley & Sons, New York (most recent supplement); and the like).

As an example, the transgene can encode a G-protein coupled receptor (GPCR), such as a GPCR from a human, a non-human mammal, a non-mammalian vertebrate, an invertebrate (e.g. an insect or nematode), a yeast, a bacteria or a plant. GPCRs are seven-transmembrane-domain polypeptides that transduce G-protein coupled signals in response to ligands. The natural ligands of different GPCRs include peptides, biogenic amines, glycoproteins, nucleotides, ions, lipids, amino acids, light and odorants. Structurally, GPCRs can be divided into three major subfamilies, each of which currently includes orphan receptors as well as receptors whose ligands are characterized (reviewed in Gether, Endocrine Reviews 21:90-113 (2000)).

Exemplary members of the Rhodopsin/β2 adrenergic receptor-like family of GPCRs include receptors for biogenic amines (adrenergic, serotonin, dopamine, muscarinic, histamine and the like), CCK, endothelin, tachykinin, neuropeptide Y, TRH, neurotensin, bombesin, growth hormone secretagogues, vertebrate and invertebrate opsins, bradykinin, adenosine, cannabinoid, melanocortin, olfactory signals, chemokines, fMLP, c5A, GnRH, eicosanoid, leukotriene, FSH, LH, TSH, fMLP, galanin, nucleotides, opioids, oxytocin, vasopressin, somatostatin and melatonin, as well as GPCRs activated by proteases.

Exemplary members of the Glucagon/VIP/Calcitonin receptor-like family of GPCRs include receptors for calcitonin, CGRP, CRF, PTH, PTHrP, glucagon, glucagon-like peptide, GIP, GHRH, PACAP, VIP, secretin and latrotoxin.

Exemplary members of the Metabotropic neurotransmitter/Calcium receptor family of GPCRs include metabotropic glutamate receptors, metabotropic GABA receptors, calcium receptors, vomeronasal pheromone receptors and taste receptors.

A database containing links to the nucleotide and amino acid sequences of numerous mammalian GPCRs, including orphan GPCRs, is available at http://www.darmstadt.gmd.de/˜gpcrdb/. The invention can be practiced with a transgene encoding any GPCR, including variants and mutants of known GPCRs, or any desired fragment thereof.

Alternatively, the invention can be practiced with a transgene that encodes a membrane protein other than a GPCR. Membrane proteins include receptors for cytokines, growth factors and hormones, including platelet-derived growth factor, epidermal growth factor, insulin, insulin-like growth factor, hepatocyte growth factor, fibroblast growth factor, interleukins, interferons and the like. Membrane proteins also include adhesion molecules, such as an integrins, cadherins and the like; immune molecules, such as antibodies and antigen-binding fragments thereof, T-cell receptors, MHC molecules, cell surface determinants and the like; ion channels; transporters; membrane proteases; death receptors; nuclear receptors; multi-drug resistant proteins; membrane cyclases; tyrosine kinases; membrane phosphatases; or gap junction proteins.

The invention can also be practiced with a transgene that encodes a polypeptide that is not normally membrane localized. For such applications, a membrane localization signal will generally be included within the transgenic polypeptide or within the ROS targeting signal, as described further below. Therefore, the transgene can encode any polypeptide of interest, such as an enzyme (e.g. a kinase, phosphatase, nuclease, protease, polymerase, and the like); binding protein (e.g. a transcription factor, docking protein, receptor agonist or antagonist, and the like); or structural protein (e.g. a cytoskeletal protein, scaffold protein and the like).

Polypeptides expressed in the ROS membrane advantageously have relatively homogeneous post-translational modifications. Accordingly, the invention can be practiced with transgenes that encode polypeptides with extensive post-translation modifications, including multiple disulfide bonds, N- or O-linked glycosylation, fatty acylation, or phosphorylation.

A suitable transgene can encode a naturally occurring polypeptide, including the exemplary polypeptides listed above, from any species of interest, such as human, non-human mammal, other vertebrate, insect, nematode, other invertebrate, plant, yeast, other eukaryote, bacteria or other prokaryote. Advantageously, the transgene can encode a polypeptide having mutations associated with human genetic diseases, such that the structural or functional consequences of these mutations can be determined.

The transgene can also encode a non-naturally occurring polypeptide, such as a polypeptide that contains one or more amino acid additions, deletions or substitutions relative to a naturally occurring sequence. Such variant polypeptides can be used, for example, to characterize the critical functional residues of the polypeptides, such as ligand and effector binding sites, and to aid in the design of suitable therapeutic ligands. Alternatively, a non-naturally occurring polypeptide can consist only of a particular fragment or domain of interest to facilitate structural or functional studies of the particular region.

A transgenic polypeptide can also optionally include additional sequences at internal, N-terminal or C-terminal positions that confer advantageous properties. Such sequences can include, for example, sequences that confer membrane localization on the polypeptide; that facilitate isolation or identification of the polypeptide; or that modulate the function, stabilize the structure or facilitate the folding of the polypeptide.

For example, as described below, a rod outer segment (ROS) targeting signal functions in conjunction with a membrane localization signal to localize a polypeptide to the ROS membrane. Accordingly, if the polypeptide does not normally contain a membrane localization signal, the transgene can be modified by recombinant methods such that the encoded polypeptide will include a membrane localization signal. Suitable membrane localization signals and methods for their use in preparing recombinant polypeptides are well known in the art, and include, for example, myristoylation signals, palmitoylation signals, farnesylation signals, prenylation signals, GPI anchor signals and transmembrane spanning sequences.

Suitable sequences that facilitate identification or isolation of a transgenic polypeptide are known in the art, and can include epitope tags (e.g. HA, myc, FLAG) for which antibodies are available or can be produced, glutathione-S-transferase, poly-His sequences, fluorescent tags (e.g. green fluorescent protein), bioluminescent tags (e.g. luciferase), and the like.

Sequences that modulate the function, stabilize the structure or facilitate the folding of a transgenic polypeptide include, for example, sequences corresponding to molecules that normally function as ligands, adaptors, effectors or scaffold molecules. By expressing these sequences as fusions with the polypeptide of interest, close proximity of the two molecules and an appropriate stoichiometry are ensured. Additionally, the fused sequences can stabilize the polypeptide in its active or inactive configuration, as desired for a particular application, allowing identification of structural features important for activation.

For example, a GPCR can be expressed as a fusion with its peptide ligand, with an arrestin, or with a G-protein α-subunit. Methods of recombinantly preparing functional GPCR-Gα fusions are known in the art (reviewed in Seifert et al., Trends Pharmacol. Sci. 20:383-389 (1999)). Constructs encoding other desired fusion proteins can be made by routine molecular biological methods.

For certain applications, it may be advantageous to construct a transgene that encodes two or more polypeptides that contain ROS targeting signals, either as separate translation products or as fusions. For example, a transgene can encode two or more different receptor polypeptides that contain ROS targeting signals, such as two or more different GPCRs. Likewise, a transgene can encode one polypeptide that contains a ROS targeting signal and another polypeptide that contains a ROS targeting signal, wherein the two polypeptides are normally associated. Thus, one polypeptide can be a ligand, adaptor, effector or scaffold molecule of the other polypeptide, as described above. For example, one polypeptide can be an arrestin or Gα subunit, while the other polypeptide is a GPCR. The two or more polypeptides can advantageously be used together, such as in screening assays described herein, or isolated from each other by methods known in the art, such as by proteolytic cleavage between fused sequences, or by immunological separation methods.

In certain embodiments, the polypeptide encoded by the transgene in the targeting constructs, cells or animals will not be a rhodopsin. As used with respect to excluded polypeptides, the term “rhodopsin” refers to a naturally occurring rhodopsin polypeptide from any species, as well as any variant or mutant forms thereof described in the art as of the priority date of the application. The term “rhodopsin” is used herein to refer either to the apoprotein, which is also known as rod opsin, and the protein with the covalently attached chromophore. An excluded rhodopsin contains the rhodopsin N-terminal amino acid sequences and the C-terminal ROS targeting signal as contiguous sequences. Unless specifically indicated, an excluded rhodopsin polypeptide is not a rhodopsin fused to a heterologous polypeptide, such as arrestin or a Gα subunit.

Examples of specifically excluded rhodopsins include wild-type Xenopus, mouse, rat, human, pig and bovine rhodopsins, as well as mutant rhodopsins that serve as animal models of retinal disorders such as retinitis pigmentosa (P23H, V20G, P27L; various C-terminal deletions and substitutions), photoreceptor degeneration (K296E), and congenital nightblindness (G90D) (Frederick et al., Invest. Opthalamol. Vis. Sci. 42:826-833 (2001); Li et al., Proc. Natl. Acad. Sci. USA 92:3551-3555 (1995); Sieving et al., J. Neurosci. 21:5449-5460 (2001); and the like).

In an invention targeting construct, the transgene is flanked by 5′ and 3′ DNA sequences that are homologous to the rhodopsin gene from the animal species of interest. Conveniently, the animal species is a mouse. However, it is contemplated that the invention can be practiced with rhodopsin genes from other species amenable to gene targeting procedures, such as rat, guinea pig, bovine, Xenopus, Zebrafish, human, pig, sheep, goat, cat, dog and non-human primate.

A flanking nucleotide sequence that is “homologous” to a rhodopsin gene sequence refers to a nucleotide sequence having sufficient identity to a rhodopsin gene sequence to allow for homologous recombination between the nucleotide sequence and an endogenous rhodopsin gene sequence in a host cell. Typically, the nucleotide sequences of the flanking homology regions are at least 90%, such as at least 95%, 98%, 99% or 100% identical to the nucleotide sequences of the endogenous rhodopsin gene to be targeted for homologous recombination. Advantageously, to enhance the homologous recombination frequency the flanking homologous regions can be isogenic with the targeted endogenous allele, which means that the DNA of the flanking regions is isolated from cells of the same genetic background as the cell into which the targeting construct is to be introduced.

Mouse rhodopsin genomic DNA sequences can be isolated from a mouse genomic DNA library, using methods known in the art (see Humphries et al., Nature Genet. 15:216-219 (1997) and Lem et al., Proc. Natl. Acad. Sci. USA 96:736-741 (1999)). Rhodopsin genomic DNA from other species can be obtained similarly. For example, a genomic library from a desired species can be screened with a probe from a rhodopsin cDNA from that species or, in view of the high degree of homology across species, a rhodopsin cDNA from another species, to isolate rhodopsin genomic DNA for use in a targeting construct. A restriction map of the genomic DNA can then be made, and suitable regions for insertion of the transgene determined.

The flanking homologous DNA sequences are of sufficient length for homologous recombination to occur between the targeting construct and an endogenous rhodopsin gene in a cell when the construct is introduced into the cell. Generally, the longer the homologous flanking sequence, the higher the efficiency of homologous recombination. An appropriate length of 5′ flanking sequence is at least about 1 kb, and is typically from about 1.5 kb to about 15 kb, such as from about 5 kb to about 10 kb. Likewise, an appropriate length of 3′ flanking sequence is at least about 1 kb, and is typically from about 1.5 kb to about 15 kb, such as from about 5 kb to about 10 kb.

The homologous sequences that flank the transgene are chosen so as direct the transgene to a desired position within the rhodopsin allele following homologous recombination. For example, if it is desired to drive expression of the transgene using native rhodopsin regulatory sequences, the 5′ homologous sequences can advantageously contain these sequences, such that the transgene will reside 3′ of the regulatory sequences in the recombined allele. The homologous regions that flank the transgene can also be chosen so as to make modifications, such as insertions, deletions and substitutions, in the recombined rhodopsin allele. For example, if it is desired to delete portions of the rhodopsin gene by homologous recombination (such as native 5′ regulatory elements, one or more exons, one or more introns), these regions are not included in the DNA sequences flanking the transgene. Deletions of portions of the endogenous rhodopsin gene are useful to ensure that a functional rhodopsin polypeptide is not expressed in the rod cells.

To provide for transcription and, ultimately, translation, of the transgene in rod cells, the construct is designed such that the transgene will be operably associated with rod-specific regulatory sequences following homologous recombination with a rhodopsin allele. As used herein, the term “operably associated” indicates that the rod-specific regulatory sequences and the transgene are positioned in such a manner so as to permit transcription of the transgene under the control of the rod-specific regulatory sequences.

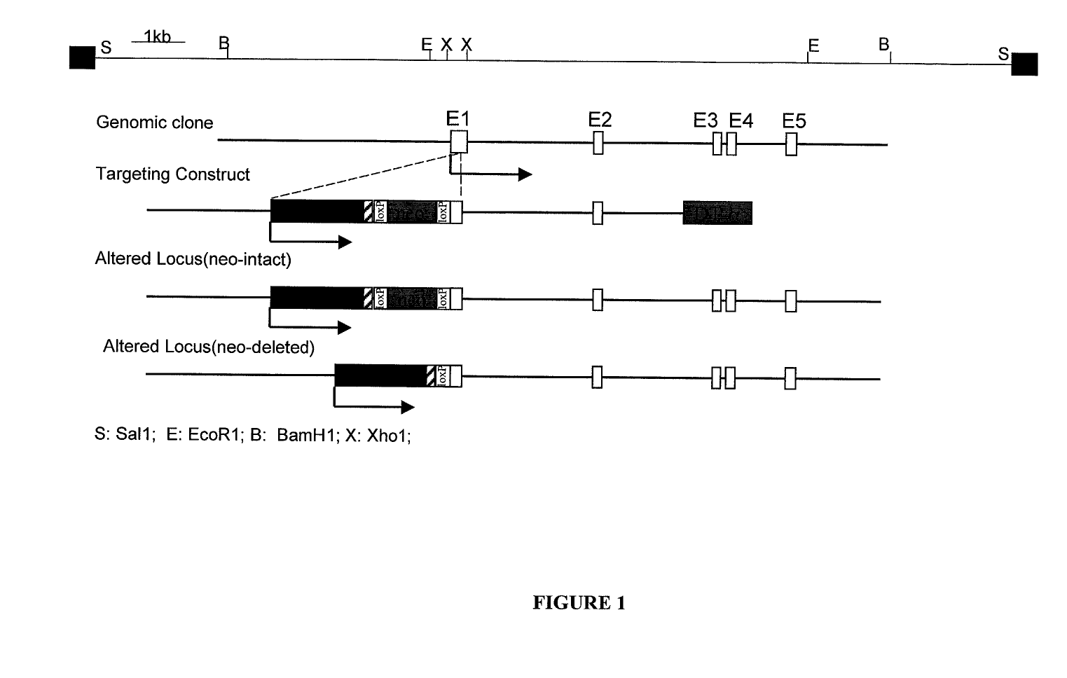

As used herein, the term “rod-specific regulatory sequences” refers to cis-acting DNA elements sufficient to direct transcription of the transgene in a rod cell. The term “rod-specific” means that the transgene is expressed at least in the rod cells, but does not require that the transgene be exclusively expressed in the rod cells. For use in a gene targeting construct, the rod-specific regulatory sequences are generally endogenous rhodopsin regulatory sequences included within the 5′ DNA sequence flanking the transgene (see FIG. 1). However, the rod-specific regulatory sequences can alternatively be rhodopsin regulatory sequences from other species, or regulatory sequences derived from other genes expressed in rod cells, such as arrestin, transducin α, β or γ subunits, phosphodiesterase α, β or γ subunits, or recoverin. Rod-specific regulatory sequences include promoter sequences that direct gene expression in the rod cells and, optionally, enhancer sequences that regulate the level of gene expression in the rod cells.

Regulatory sequences from rhodopsin genes are recognized by trans-acting factors in rod cells across species. For example, both bovine and human rhodopsin regulatory elements have been shown to direct expression of trangenes to mouse photoreceptor cells (Zack et al., Neuron 6:187-199 (1991); Nie et al., J. Biol. Chem. 271:2667-2675 (1996)). Rod-specific regulatory sequences can thus include regulatory elements from a rhodopsin from any vertebrate species (e.g. mouse, other rodent, bovine, Xenopus, human, pig, sheep, cat, dog, non-human primate, Zebrafish) and can include non-native DNA sequences.

Rhodopsin regulatory sequences, including promoter and enhancer elements, have been characterized in a number of species, including Xenopus (Mani et al., J. Biol. Chem. 28:36557-36565 (2001)), mouse (Lem et al., Neuron 6:201-210 (1991)) and bovine (Nie et al., J. Biol. Chem. 271:2667-2675 (1996). These studies have indicated that fragments from −2174 to +70 bp; from −735 to +70 bp; from −222 to +70 bp; and from −176 to +70 bp, relative to the bovine rhodopsin mRNA start site, are able to direct photoreceptor-specific gene expression in transgenic mice (Nie et al., supra (1996)), indicating that the minimal cell-specific promoter lies within the region −176 to +70 bp of the bovine rhodopsin transcription start site. Likewise, 4.4 kb and 0.5 kb fragments from the mouse rhodopsin gene are able to direct photoreceptor-specific gene expression in transgenic mice (Lem et al., supra (1991)), indicating that the minimal cell-specific promoter lies within about 500 bp 5′ of the mouse rhodopsin transcription start site. Additionally, a highly conserved region of about 102 bp about 2 kb 5′ of the transcription start site of the bovine, human, mouse and rat rhodopsin genes has been identified as a transcription enhancer region (Nie et al., supra (1996)).

If desired, rod-specific regulatory elements can be modified from a native sequence to enhance tissue specificity or expression levels. For example, negative regulatory elements can be deleted so as to increase expression levels, without a change in rod cell specificity (Mani et al., supra (2001)). Additionally, multiple copies of enhancer elements can optionally be included, and sequences between the promoter and enhancer elements can optionally be deleted. Based on knowledge of rod-specific positive and negative regulatory elements, a skilled person can determine an appropriate sequence for directing expression of a transgene to rod cells.

A convenient assay for confirming that a particular regulatory sequence directs rod-specific gene expression takes advantage of the ease with which transgenic Xenopus can be made. A detectable reporter gene, such as green fluorescent protein or luciferase (or the desired transgene), can be operably linked to the candidate rod-specific regulatory sequence, and the construct transfected into fertilized Xenopus embryos by standard methods. Expression of the reporter gene (or the desired transgene) in the rod cells of the resulting tadpoles confirms that the regulatory sequence directs rod-specific gene expression (see Mani et al., supra (2001)).

The polypeptide expressed by the transgene also contains a rod outer segment (ROS) targeting signal to localize the polypeptide to the ROS membrane. Vertebrate rod cells consist of an outer segment that contains stacks of rhodopsin-containing disc membranes connected to the inner segment by a ciliary process. The inner segment contains the metabolic machinery of the cells, such as the mitochondria and Golgi. As used herein, the term “rod outer segment targeting signal” refers to a peptide sequence that confers localization of a heterologous polypeptide to the ROS membrane. An acceptable ROS targeting signal does not need to confer localization of the polypeptide exclusively to the ROS membrane. A small amount of expression of the polypeptide in other parts of the rod cell, including the inner segment, nucleus or synaptic body, will not be detrimental, so long as the polypeptide is abundantly expressed in the ROS membrane.

The necessary and sufficient features of vertebrate ROS targeting signals have been determined in transgenic Xenopus laevis by expressing chimeras between heterologous polypeptides and regions of X. laevis rhodopsin under the control of the X. laevis rhodopsin promoter. These studies have revealed that the C-terminal 8 amino acids of X. laevis rhodopsin (SSSQVSPA; SEQ ID NO: 1)) are sufficient to confer outer segment membrane targeting on a heterologous polypeptide containing membrane association signals. A peptide containing the C-terminal 25 amino acids of X. laevis rhodopsin (DEDGSSAATAKTEASSVSSSQVSPA; SEQ ID NO: 2) also effectively confers outer segment membrane targeting on a heterologous polypeptide containing membrane association signals. These sequences were not sufficient, however, to confer ROS targeting on a cytoplasmic polypeptide (Tam et al., J. Cell Biol. 151:1369-1380 (2000)).

A longer sequence that contains the di-cysteine palmitoylation signal of rhodopsin, such as the C-terminal 44 amino acids of X. laevis rhodopsin (KQFRNCLITTLC*C*GKNPFGDEGGSSAATSKTEASSVSSSQVSPA; SEQ ID NO: 3), is able to confer outer segment membrane targeting on a polypeptide that does not have its own membrane association sequences (Tam et al., supra (2000)). The two cysteine residues that are palmitoylated in the X. laevis C-terminal ROS sequence are indicated by asterisks.

ROS targeting signals can be recognized across species. For example, human rhodopsin can functionally rescue murine rod photoreceptors in rhodopsin knock-out mice (McNally et al., Hum. Mol. Genet. 8:1309-1312 (1999)). Therefore, a ROS targeting signal can be a naturally-occurring ROS from a rhodopsin from any vertebrate species (e.g. mouse, other rodent, bovine, Xenopus, human, pig, sheep, cat, dog, non-human primate, Zebrafish) or can be a non-naturally occurring sequence. The sequences of rhodopsins from a variety of species are known in the art (see, for example, GenBank gi:129207 (human); gi:223659 (bovine); gi:129210 (mouse)). A ROS targeting signal can thus contain the native C-terminal sequence from a rhodopsin from any vertebrate species (e.g. mouse, other rodent, bovine, Xenopus, human, cat, dog, non-human primate; Zebrafish) or can be a non-naturally occurring sequence, such as a consensus sequence determined by aligning the ROS sequences from numerous species.

For example, a ROS targeting signal can include the eight (8) (ETSQVAPA; SEQ ID NO: 4) or nine (9) (TETSQVAPA; SEQ ID NO: 5) C-terminal residues shared by mouse, human and bovine rhodopsin, which are recognized by the rho1D4 monoclonal antibody (Molday et al., Biochemistry 22:653-660 (1983); MacKenzie et al., Biochemistry 23:6544-6549 (1994); Molday et al., Biochemistry 24:776-781 (1985)). Expression of a transgenic polypeptide containing the 1D4 epitope as the ROS targeting signal can advantageously be detected, and the polyepeptide isolated, by standard immunological assays using the rho 1D4 antibody. Another convenient ROS targeting sequence contains the 15 C-terminal residues from bovine rhodopsin (STTVSKTETSQVAPA; SEQ ID NO: 6). Other suitable ROS targeting sequences correspond to the C-terminal amino acids (such as from about 8 to about 50 amino acids) of a vertebrate rhodopsin.

As described above with respect to rod-specific regulatory elements, a convenient assay for confirming the function of a candidate ROS targeting signal is to prepare transgenic Xenopus expressing the polypeptide/ROS fusion (and optionally further containing a detectable moiety) in their rod cells, and observing localization of the transgenic polypeptide to the rod outer segment membranes by microscopy (see, for example, Moritz et al., J. Biol. Chem. 276:28242-28251 (2001); and Tam et al., supra (2000)).

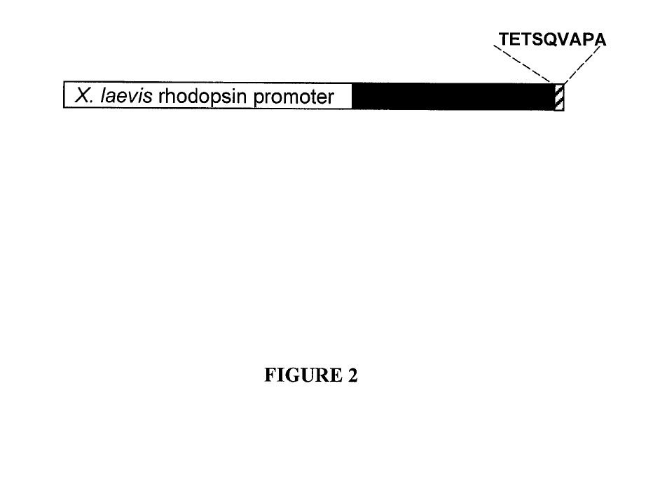

As an example, as shown in FIG. 2, the Xenopus laevis rhodopsin gene promoter can be inserted in front of a nucleotide sequence encoding the full-length human cannabinoid receptor 2 (CB2) fused to the 9 amino acid ROS targeting signal shown (SEQ ID NO: 5). The construct can be transfected into Xenopus embryos and polypeptide expression in the ROS membrane of the tadpole confirmed by immunolocalization with a CB2 antibody.

A gene targeting construct can also contain one or more selectable markers. The construct generally contains at least one positive selection marker, the presence of which in the genome of a targeted cell indicates insertion of the construct into the genome, which can be random insertion or insertion by homologous recombination. Advantageously, the construct can also contain a negative selection marker, generally positioned at the 5′ or 3′ end of a linearized targeting construct, outside of the region of homology. The absence of the negative selection marker in the genome of the targeted cell, together with the presence of the positive selection marker, enriches for cells in which the construct has likely been inserted into the genome by homologous recombination rather than by random integration. Suitable positive and negative selection markers for gene targeting constructs can be selected by the skilled person, and methods for their use are well known in the art.

Positive selection markers include expressible genes that confer survival on a cell, such as genes that confer resistance to the drugs neomycin, hygromycin, puromycin or histidinol resistance. Alternatively, since ES cell lines are available that are deficient for hypoxanthin-phosphoribosyltransferase (HPRT), an expressible HPRT gene can serve as a positive selection marker and transfectants selected in HAT medium (Muller, Mech. Devel. 82:3-21 (1999)).

Negative selection markers include expressible genes that are directly or indirectly toxic to a cell. An exemplary negative selection marker is an expressible gene encoding the diphtheria toxin-A fragment (DTα). Another negative selection marker is the herpes simplex virus thymidine kinase (tk) gene that confers sensitivity to toxic nucleoside analogs such as gancyclovir or FIAU. An alternative negative selection marker is an expressible gene whose product can be recognized by an immunotoxic conjugate, such as the IL-2 receptor gene whose product is recognized by the recombinant immunotoxin anti-Tac (Fv)-PE40 (Muller, supra (1999); Kobayashi et al., Nucleic Acids Res. 24:3653-3655 (1996)).

In the exemplary gene targeting construct shown in FIG. 1, the homologous sequence 5′ of the transgene (which encodes a GPCR containing a C-terminal ROS targeting signal) contains about 1-5 kb of the mouse rhodopsin gene, including the native 5′ regulatory elements. The homologous sequence 3′ of the transgene contains about 1-5 kb, including part of exon 1 and optionally exon 2, of the mouse rhodopsin gene. The total length of the 5′ and 3′ homologous sequences are generally between 4 and 8 kb. The 5′ and 3′ homologous sequences are generally 1.5 kb or greater, and more usually 2 kb or greater, with the length depending, in part, on the availability of appropriate restriction sites. The construct also contains an expressible diphtheria toxin A gene (DTα) as a negative selection marker, and an expressible floxed neo gene 3′ of the trangene as a positive selection marker. Following homologous recombination between this construct and a mouse rhodopsin allele, the transgene and the floxed neo sequence will be inserted 3′ to the rhodopsin regulatory sequences so as to delete a portion of exon 1. The native mouse rhodopsin regulatory sequence thus directs expression of the transgene in rod cells, and the ROS targeting signal provides for localization of the encoded polypeptide in the ROS membrane. The insertion of the transgene at the rhodopsin allele functionally disrupts rhodopsin gene expression. Therefore, in an animal homozygous for the targeted allele, rhodopsin is expressed at low or undetectable levels, and the transgene is expressed in the ROS membrane.

The invention also provides a vector containing the gene targeting construct, and a host cell containing the gene targeting construct. A suitable vector can be a plasmid, cosmid, phage, BAC or other cloning vector into which large pieces of DNA can be inserted. The vector generally contains an origin of replication for amplifying the construct in a host cell. The vector can advantageously also contain a selection marker for selecting for host cells containing the vector. For amplifying the vector, the host cell will typically be a bacterial cell, but can alternatively be a yeast, insect, or mammalian cell. Methods of introducing a vector into a host cell are well known in the art (see, for example, Sambrook et al., supra (2001); Ausubel et al. supra (most recent supplement)).

Vectors suitable for use in gene targeting applications are available commercially (e.g. from Stratagene and Lexicon Genetics, Inc.). Dedicated gene targeting vectors conveniently include positive and negative selection markers suitable for use in mammalian cells, together with appropriate cloning sites for inserting homologous gene sequences and transgenic sequences.

For certain applications, it is desirable to be able to remove the positive selection marker from the genome of a targeted cell or transgenic animal. Accordingly, a gene targeting construct can contain a positive selection marker operably positioned with respect to one or more sequences that facilitate its excision from the genome. Sequences suitable for facilitating excision of specific DNA sequences include recognition sites for site-specific recombinases. A variety of site-specific recombinases, including enzymes from bacteriophage, bacteria and yeast, and their recognition sites are known in the art (reviewed in Kilby et al., Trends in Genet. 9:413-421 (1993)). Those skilled in the art can choose appropriate sequences and corresponding enzymes for selective removal of the positive selection marker.

An exemplary system for specific DNA excision is the Cre/lox recombination system. The Cre/lox recombination system involves the use of the site-specific recombinase Cre (causes recombination) from phage P1 that recognizes and binds to a 34-bp long, partly palindromic target sequence called loxP (locus of crossover x in P1). The loxP sequence is set forth as SEQ ID NO: 7 (5′-ATAACTTCGTATAGCATACATTATACGAAGTTAT-3′). Cre recombinase has the ability to efficiently excise, by intramolecular recombination, any sequence placed between two loxP sites in the same relative orientation. A DNA sequence between two loxP sites in the same relative orientation is called a “floxed” sequence. As a result of Cre activity, one loxP site remains within the genome and one loxP site is is found on the excised circularized fragment (see FIG. 1; for reviews, see Muller, supra (1999); and Kilby et al., supra (1993)).

Methods are known in the art to excise a floxed DNA sequence, such as a floxed positive selection marker, from a targeted allele. One method is to transiently express Cre from an expression cassette in targeted embryonic stem (ES) cells, followed by screening ES clones to confirm deletion of the floxed sequence (see Xu et al., Genesis 30:1-6 (2001); Gu et al., Science 265:103-106 (1994)). An alternative method is to cross a transgenic mouse whose genome contains a floxed sequence with a transgenic mouse carrying the EIIa-Cre gene (Xu et al., supra (2001); Lakso et al., Proc. Natl. Acad. Sci. USA 93:5860-5865 (1996)). A further alternative method is to inject a Cre-expression plasmid into the pronuclei of fertilized eggs from transgenic animals bearing the floxed sequence (Xu et al., supra (2001)). In the latter two methods, progeny mice in which the floxed sequence is deleted are identified by screening. Those skilled in the art can determine additional methods of removing a floxed sequence from a targeted allele.

The invention also provides a cell whose genome contains a functional disruption of one or both endogenous rhodopsin alleles, and further contains a transgene encoding a polypeptide comprising a ROS targeting signal operably associated with a rod-specific regulatory sequence. Also provided is an animal whose genome contains a functional disruption of one or both endogenous rhodopsin alleles, and further contains a transgene encoding a polypeptide comprising a ROS targeting signal operably associated with a rod-specific regulatory sequence.

In certain embodiments, the cell is a mouse cell and the animal is a mouse. However, it is contemplated that the invention can be practiced with other species amenable to gene targeting procedures, such as rat, guinea pig, bovine, Xenopus, human, pig, sheep, goat, cat, dog, non-human primate or Zebrafish.

As used herein, the term “functional disruption” with respect to a rhodopsin allele means that the allele contains a mutation that prevents the normal function of the encoded polypeptide, such as a mutation that prevents expression of a normal rhodopsin polypeptide or that prevents expression of normal amounts of the rhodopsin polypeptide. The terms “functional disruption” and “knockout” are used herein synonymously. The mutation causing the functional disruption can be an insertion, deletion or point mutation.

In one embodiment, both rhodopsin gene alleles are functionally disrupted such that expression of the rhodopsin gene product is substantially reduced or substantially absent in cells of the animal. The term “substantially reduced” is intended to mean that less than 50% of the normal amount of rhodopsin is produced in rod cells of the animal, whereas the term “substantially absent” is intended to mean that essentially undetectable amounts of rhodopsin are produced in rod cells of the animal. Although animals with substantially reduced or substantially absent levels of rhodopsin are typically made by disrupting the coding region of the rhodopsin gene, an alternative approach is to disrupt the cis-regulatory elements of the gene such that transcription of the gene is down-regulated.

The skilled person will appreciate that there are various methods of making a cell or animal whose genome contains both a functional disruption of the rhodopsin gene and a particular transgene. For example, such a cell or animal can be obtained as a result of homologous recombination between a gene targeting construct containing the transgene and the endogenous rhodopsin gene, such that the transgene is inserted into a rhodopsin allele (called a “gene knock-in”). Alternatively, such a cell or animal can be obtained as a result of random insertion of the transgene into a rhodopsin gene knockout background, either directly or by cross-breeding a transgenic animal with a knockout animal.

An invention cell is intended to include a cell obtained prior to implantation into the animal (such as an embryonic stem cell, germ cell or embryo cell); a cell as it exists in the transgenic animal or its progeny; and a cell obtained or derived from the transgenic animal or progeny of said cell, such as an organ, tissue, isolated primary cell or established cell line.

An invention cell optionally expresses the transgenic polypeptide. For example, the cell can be a rod cell as it exists in a transgenic animal, or a rod cell isolated from a transgenic animal or progeny of said cell, such as an established rod cell line. Rod cells isolated from the transgenic animals of the invention generally express the transgenic polypeptide, due to the rod-specific regulatory elements directing transcription in the rod cells, as well as the ROS targeting signal which localize the polypeptide to the ROS membrane.

Suitable gene targeting constructs for use in a knock-in approach have been described above. Methods for preparing cells and animals using a gene targeting construct are well known in the art. Briefly, the targeting construct is introduced into an appropriate acell, such as an embryonic stem cell, by any of several techniques known in the art, including electroporation, calcium phosphate precipitation, DEAE-dextran transfection, microinjection, lipofection and the like. The cell is then cultured for a period of time and under conditions sufficient to allow for homologous recombination between the introduced targeting construct and an endogenous rhodopsin gene. Cells containing the inserted DNA are identified, such as by the positive or positive/negative selection methods described above. The selected cells can then be screened for homologous recombination at the endogenous rhodopsin gene locus by standard techniques, such as Southern hybridization or PCR using a probe or primer pair which distinguishes the endogenous allele from the recombinant allele.

If it is desired to create a cell homozygous for the rhodopsin gene disruption without a breeding step, drug escalation can be used (Mortensen et al., Mol. Cell. Biol. 12:2391-2395 (1992)) on the heterozygous cells. Alternatively, the first allele of a wild type cell can be disrupted by a first homologous recombination event that is selected with one marker (e.g. neomycin resistance) and then the second allele of the heterozygous cells can be disrupted by a second homologous recombination event that is selected with a different marker (e.g. hygromycin resistance).

To create a knock-in animal, an embryonic stem (ES) cell containing the recombinant allele is introduced into a blastocyst or aggregated with a morula, the blastocyst or morula is implanted into a pseudopregnant foster mother, and the embryo allowed to develop to term. The resultant animal is a chimera having cells descended from the embryonic stem cell. Chimeric animals in which the embryonic stem cell has contributed to the germ cells of the animal can be mated with wild type animals to produce animals heterozygous for the knock-in gene in all somatic and germ cells. The heterozygous animals can then be mated to create homozygous animals. Methods for obtaining, culturing and manipulating ES cells and other suitable cells for homologous recombination, and for preparing and identifying animals that are chimeric, heterozygous or homozygous for the recombinant allele, are known in the art and reviewed, for example, in Sedivy et al., Gene Targeting, W. H. Freeman and Co., New York (1992); Joyner (ed.) Gene Targeting: a Practical Approach. Oxford University Press, New York, 2^{nd} ed. (1998); and Ledermann, Exp. Physiol. 85:603-613 (2000)).

As an alternative to a knock-in strategy, the cells and animals of the invention can be made by introducing an appropriate transgenic construct into a genetic background in which the rhodopsin gene is functionally disrupted. Lines of mice with functional disruptions of the rhodopsin gene have been described in the art (see, for example, Humphries et al., Nature Genet. 15:216-219 (1997); Lem et al., Proc. Natl. Acad. Sci. USA 96:736-741 (1999)), and offspring of these mice can be obtained or additional lines of knockout animals prepared by similar methods.

A suitable construct for insertion of a transgene contains a DNA sequence encoding the transgenic polypeptide and ROS signal, operably linked to rod-specific regulatory sequences. Suitable polypeptides, ROS signals and rod-specific regulatory sequences have been described above. An exemplary rod-specific regulatory sequence for use in a transgenic construct is a 2.1 kb 5′ HindIII fragment from mouse rhodopsin (Geiger et al., Invest. Opthamol. Vis. Sci. 35:2667-2681 (1994)).

Methods for preparing transgenic animals are well known in the art. As an example of a typical method, the transgenic DNA construct is introduced into the male pronucleus of a fertilized egg (zygote), which is then implanted into a pseudopregnant female recipient animal. The embryo is grown to term, and offspring containing the transgene (heterozygous founder animals) are identified by Southern blotting or PCR. Different founder animals will have different sites of transgene integration, which can affect gene expression. Lines of animals with suitable expression of the transgenic polypeptide in rod cells can be identified and bred with wild-type animals to produce more animals with the same insertion (see Sedivy et al., supra (1992); Hogan et al., Manipulating the Mouse Embryo: A Laboratory Manual, 2nd ed., Cold Spring Harbor Laboratory (1994)).

Alternative methods known in the art can be used to introduce a transgene into animals to produce the founder lines of transgenic animals (see, for example, Hogan et al., supra, 1994; U.S. Pat. Nos. 5,602,299; 5,175,384; 6,066,778; and 6,037,521). Such methods include, for example, retrovirus mediated gene transfer into germ lines (Van der Putten et al., Proc. Natl. Acad. Sci. USA 82:6148-6152 (1985)); electroporation of embryos (Lo, Mol Cell. Biol. 3:1803-1814 (1983)); and sperm-mediated gene transfer (Lavitrano et al., Cell 57:717-723 (1989)).

To make an animal with a transgene in a rhodopsin knockout background, generally a transgenic animal will be crossed with a knockout animal. Alternatively, the transgene can be introduced into a zygote containing the rhodopsin knockout allele, and the zygote grown to term as described above. By either method, offspring of the desired genotype are identified and additional animals produced by breeding.

The invention animals, whose genome contains a functional disruption of one or both endogenous rhodopsin alleles, and further contains a transgene encoding a polypeptide comprising a ROS targeting signal operably associated with a rod-specific regulatory sequence, can advantageously be used in a variety of applications. For example, large quantities of substantially purified transgenic polypeptide can be isolated from the outer segment membrane of rod cells of the eyes of the animals. For such purposes, animals in which the expression of endogenous rhodopsin is substantially absent due to functional disruption of both endogenous rhodopsin alleles are preferred, so that contamination of the ROS membrane with rhodopsin is minimized and purification is simplified. Additionally, intact rod cells and extracts thereof containing the transgenic polypeptide can be used in applications described herein.

In normal animals, about 90% of the protein content of the rod outer segment disc membranes is rhodopsin. In invention animals, due to rod cell-specific expression of the transgene and inclusion of the ROS targeting signal in the encoded polypeptide, it is expected that a substantial proportion (such as at least 10%, 25%, 50%, 75% or more) of the protein content of the rod outer segment disc membranes will instead be the transgenic polypeptide.

In normal animals, the typical yield of purified rhodopsin is about 0.1-1.0 nmol per mouse eye (Li et al., Proc. Natl. Acad. Sci. USA 92:3551-3555 (1995); Van Hooser et al., Proc. Natl. Acad. Sci. USA 97:8623-8628 (2000)). In invention animals, it is expected that a similar amount of transgenic polypeptide can be prepared from a similarly sized eye, with the actual amount depending on the animal species.

The skilled person can determine an appropriate method of substantially purifying a transgenic polypeptide from the rod cells of an invention animal. Generally, retinas are dissected from a suitable number of animals, and rod outer segments isolated as described by Papermaster et al., Methods Enzymol. 81:48-52 (1982) or Okada et al., Photobiol. 67:495-499 (1998). For example, retinas can be homogenized in a sucrose buffer, crude ROS sedimented by low-speed centrifugation, and substantially purified ROS isolated by density gradient centrifugation. For certain applications, it may be more convenient to use rod cell extracts, retinal extracts, or eye extracts as the starting source for substantially purifying the transgenic polypeptide.

The transgenic polypeptide can be solubilized from the ROS membrane using a suitable detergent. Solubilization conditions can advantageously be optimized so as to provide for single-step purification of the polypeptide. For example, alkyl(thio)glucosides with an appropriate hydrophilic-lipophilic balance (e.g. octylthioglucoside) in combination with a divalent cation provided for single-step purification of rhodopsin from ROS (Okada et al., supra (1998)). Alternatively, the solubilized polypeptide can be subjected to further purification using standard biochemical and immunological procedures, which can be chosen by the skilled person depending, for example, on the biological and immunological properties of the polypeptide and the degree of purity required for a particular application. Advantageously, a polypeptide containing a ROS targeting signal that contains the 1D4 epitope can be recognized by the 1D4 monoclonal antibody. Accordingly, the transgenic polypeptide can be isolated by standard immunoaffinity procedures known in the art.

The transgenic polypeptide can be obtained in sufficient concentration and purity so as to use to prepare a crystal suitable for structural analysis by X-ray crystallography. As described previously, as opposed to polypeptides expressed in tissue culture, polypeptides expressed in the ROS are relatively homogeneous with respect to post-translational modifications, which greatly facilitates crystallization. The conditions for generating high-quality crystals will depend on the polypeptide itself. However, exemplary conditions for preparing crystals from rhodopsin are described in Okada et al., J. Structural Biol. 130:73-80 (2000), and are expected to be relevant to many trangenic GPCRs as well as other transgenic polypeptides isolated from ROS membranes. Briefly, crystals can be prepared by hanging drop vapor diffusion from a solution containing at least about 5 mg/ml polypeptide in buffer containing about 30 mM MES or sodium acetate, 5-7 mM P-mercaptoethanol, 65-90 Zn(OAc)₂, 0.55-0.75% HTPO, 0.45%-0.55% nonyl glucoside and 0.84-0.86 M ammonium sulfate. Alternative crystallization buffers and additives that can be used to improve crystallization are known in the art (see, for example, Rees et al., eds., Protein Engineering: A Practical Approach, Oxford University Press, Oxford (1992)).

A substantially purified transgenic polypeptide can also be used to prepare antibodies. Such antibodies can be advantageous in recognizing the polypeptide in its native form and with its native post-translational modifications. Optionally, for such purposes the transgenic polypeptide can be conjugated to a carrier protein and/or formulated together with an adjuvant to increase its immunogenicity, and used to immunize an appropriate animal. Methods of preparing polyclonal and monoclonal antibodies and antigen-binding fragments thereof (e.g. VL, VH and Fd; monovalent fragments, such as Fv, Fab, and Fab′; bivalent fragments such as F(ab′)₂; single chain Fv (scFv); and Fc fragments) and the like are described, for example, in Harlow and Lane, Antibodies: A Laboratory Manual, Cold Spring Harbor Laboratory, New York (1989); in Day, E. D., Advanced Immunochemistry, Second Ed., Wiley-Liss, Inc., New York, N.Y. (1990); and in Borrebaeck (Ed.), Antibody Engineerinq, Second Ed., Oxford University Press, New York (1995).

A further application for substantially purified transgenic polypeptides is in the preparation of pharmaceuticals. For example, if the transgenic polypeptide is an antibody, it can be conjugated to a toxin and administered to an individual to specifically target cells expressing the corresponding antigen, such as tumor cells. As a further example, if the transgenic polypeptide is a receptor agonist or antagonist, it can be administered to an individual to modulate receptor signaling associated with a pathological condition. Pharmaceutical applications for various polypeptides are known in the art or can be determined. The substantially purified polypeptide can be formulated together with a pharmaceutically acceptable excipient. The amount of polypeptide and the precise formulation will depend on the nature and biological activity of the polypeptide, as well as the intended route of administration. Suitable methods and excipients for formulating pharmaceuticals are desribed, for example, in Remington's Pharmaceutical Sciences (Maack Publishing Co., Easton, Pa., most recent edition).

The transgenic polypeptide can also be used in drug screening applications. For example, rod cells, ROS membrane extracts, or substantially purified polypeptides can be contacted with a candidate compound, and the ability to the compound to bind the polypeptide determined. A compound that binds the polypeptide is a candidate ligand, agonist, antagonist or reverse agonist of the polypeptide. The functional effect of the compound can subsequently be determined by functional assays appropriate to the particular polypeptide. Suitable candidate compounds for use in screening assays include chemical or biological molecules such as simple or complex organic molecules, metal-containing compounds, carbohydrates, peptides, proteins, peptidomimetics, glycoproteins, lipoproteins, nucleic acids, antibodies, and the like, and libraries of such compounds can readily be prepared or are commercially available. Various binding assays, including high-throughput binding assays are known in the art and can be used in screening assys, including scintillation proximity assays (SPA), UV or chemical cross-linking, competition binding assays, biomolecular interaction analysis (BIA), surface plasmon resonance (SPR), mass spectrometry (MS), nuclear magnetic resonance (NMR), and fluorescence polarization assays (FPA). The skilled person can determine appropriate compounds and assays for a particular screening application.

Intact cells from a transgenic animal that express the transgenic polypeptide, including rod cells within the animal retina and rod cells isolated from the animal, can also be used in drug screening assays, including binding assays similar to those described above and function-based screening assays. Appropriate function-based screening assays will depend on the normal function of the polypeptide. For example, if the transgenic polypeptide is a receptor, signaling through the receptor in response to the compound can be determined. Exemplary signaling assays depend on the nature of the receptor, but can include, for example, determining altered production or turnover of a second messenger, NTP hydrolysis, influx or efflux of ions or amino acids, altered membrane voltage, increased or decreased protein phosphorylation, altered activity of an enzyme, altered protein-protein interactions, relocalization of a protein within the cell, or induction of gene expression. For certain functional assays in which the relevant effector molecules or reporter genes are not normally present in rod cells, the animal genome can be further modified by knock-in or transgenic methods so as to express these components. The effect of naturally occurring and man-made mutations on transgenic polypeptide activity can likewise be determined by function-based assays.

Additional applications for the transgenic animals, cells and substantially purified transgenic polypeptides of the invention can be determined by those skilled in the art.

The following examples are intended to illustrate but not limit the present invention.

### EXAMPLE I

This example shows the construction of a gene targeting construct to replace mouse rhodopsin in the retina with a G-protein coupled receptor.

A genomic fragment containing all five exons of mouse rhodopsin and its regulatory elements is obtained by the method described in Humphries et al., Nature Genetics 15:216-219 (1997). Briefly, a rhodopsin cDNA probe is used to isolate a clone containing a 129Sv-derived mouse genomic fragment from a λ phage library. A restriction map of this fragment showing relevant restriction sites is shown in FIG. 1 (top). An 11 kb BamH1 fragment derived from the initial genomic fragment is subcloned into a pKO Scrambler V907 vector (Lexicon Genetics, Inc.) to generate the genomic clone shown in FIG. 1.

A transgenic cassette containing a G-protein coupled receptor cDNA tagged at its C-terminus with a 1D4 tag and a neomycin resistance gene flanked by two loxP sites is first constructed by standard molecular biology methods. Briefly, by PCR the termination codon of the GPCR cDNA is replaced by a sequence encoding, in-frame, the 9 amino acid 1D4 epitope tag (TETSQVAPA; SEQ ID NO: 5) followed by a stop codon.

The tagged GPCR is ligated to the ploxp-neo-loxP (“floxed neo”) cassette prepared as described in Yang et al., Proc. Natl. Acad. Sci. USA 95:3667-3672 (1998). The GPCR/floxed neo cassette is then ligated into the rhodopsin genomic clone between the rhodopsin promoter and exon 1, deleting part of the exon 1 coding sequence, such that expression of the GPCR is driven by the rhodopsin promoter.

A diphtheria toxin A chain cassette (Labarca et al., Proc. Natl. Acad. Sci. USA 98:2786-2791 (2001)) is also ligated at the 3′ end of the construct to provide the targeting construct shown in FIG. 1. The construct thus contains both an excisable positive selection marker (neo) and a negative selection marker (DTα) for use in selecting for homologous recombination in ES cells.

Targeting constructs suitable for replacing the mouse rhodopsin gene with other transgenes are made by similar methods.

### EXAMPLE II

This example shows the construction of a gene targeting construct to replace mouse rhodopsin in the retina with the human cannabinoid receptor 2.

The human cannabinoid receptor 2 (CB2) cDNA (Genbank Accession No. X74328) is cloned from a human spleen cDNA library. The 9 amino acid 1D4 tag is added to the C-terminus of CB2 by PCR using the sense primer 5′-GCC GCC ACC ATG GAG GAA TGC TGG GTG AC (SEQ ID NO: 8) and the anti-sense primer 5′-TTA GGC TGG AGC CAC CTG GCT GGT CTC CGT CTT GGA AGC GGT GGC AGA G (SEQ ID NO: 9). The junction is sequenced to confirm that the CB2/1D4 fusion is in-frame. A neomycin resistance cassette (neo), with a phosphoglycerate kinase promoter and polyadenylation signal and flanked by loxP sites, is inserted downstream of the CB2/1D4 fusion. The targeting constructed is created by replacing the DNA segment between the Xho1 sites of the rhodopsin gene with the CB2-neo cassette, deleting 15 bp upstream of the translation start site and the first 111 codons of the rhodopsin gene. The diphtheria toxin A chain (DTα) gene with the RNA polymerase II promoter is inserted at the 3′end of the targeting construct to provide negative selection.

### EXAMPLE III

This example shows the introduction of a gene targeting construct into embryonic stem (ES) cells and the production of transgenic mice.

The gene targeting construct described in Example I or II is electroporated into 129Sv ES cells, and the ES cells are cultured in the presence of the neomycin analog G418. Correctly targeted ES clones, which have an altered rhodopsin locus as shown in FIG. 1 or 2, are resistant to G418. Incorrectly targeted clones are killed by expression of the DTα gene. DNA from G418 resistant clones is screened to confirm homologous recombination, by PCR analysis and Southern blotting.

The ES cells are transiently transfected with a Cre recombinase expression vector, such as a cytomegalovirus-Cre plasmid, and Cre-mediated excision of the neo^{r} gene at the flanking lox sites confirmed by sequence analysis of the PCR-amplified gene segment.

Correctly targeted ES cell clones with the neo^{r} gene excised are microinjected into C57BL/6 blastocysts, which are then implanted into pseudopregnant female mice. Chimeric male offspring are identified by their mixed coat color and bred to females, and offspring heterozygous for the targeted allele identified by PCR analysis and Southern blotting. Heterozygotes are then cross-bred to produce homozygous mice.

The homozygous mice produce the transgenic polypeptide instead of rhodopsin in the outer segment membrane of rod cells.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention.

What is claimed is:

1. A gene targeting construct, comprising
a transgene encoding a polypeptide comprising a rod outer segment (ROS) targeting signal, said transgene flanked by 5′ and 3′ DNA sequences which are homologous to the mouse rhodopsin gene,
wherein homologous recombination between said construct and a mouse rhodopsin gene results in operable association between said transgene and a rod-specific regulatory sequence.

2. The construct of claim 1, wherein said polypeptide is a G protein-coupled receptor (GPCR).

3. The construct of claim 2, wherein said GPCR is a cannabinoid receptor.

4. The construct of claim 1, wherein said polypeptide is a fusion protein.

5. The construct of claim 1, wherein said ROS targeting signal comprises SEQ ID NO: 4.

6. The construct of claim 1, further comprising a positive selection marker.

7. The construct of claim 6, wherein said positive selection marker is a neomycin resistance gene.

8. The construct of claim 6, wherein said positive selection marker is flanked by loxP sites.

9. The construct of claim 1, further comprising a negative selection marker.

10. The construct of claim 9, wherein said negative selection marker is a diphtheria toxin A fragment gene.

11. The construct of claim 1, wherein said rod-specific regulatory sequence comprises a rhodopsin promoter.

12. The construct of claim 1, wherein the 5′ flanking DNA sequence comprises a mouse rhodopsin promoter.

13. The construct of claim 1, wherein the 3′ flanking sequence comprises a portion of exon 1 of mouse rhodopsin.

14. The construct of claim 1, wherein the 3′ flanking sequence comprises exon 2 of mouse rhodopsin.

15. A vector comprising the construct of claim 1.

16. A cell comprising the construct of claim 1.

17. A mouse cell whose genome comprises:
a) a functional disruption of one or both endogenous rhodopsin gene alleles, and
b) a transgene encoding a polypeptide comprising a ROS targeting signal operably associated with a rod-specific regulatory sequence,
wherein said polypeptide is not a rhodopsin.

18. The cell of claim 17, wherein said polypeptide is a GPCR.

19. The cell of claim 18, wherein said GPCR is a cannabinoid receptor.

20. The cell of claim 17, wherein said polypeptide is a fusion protein.

21. The cell of claim 17, wherein said ROS targeting signal comprises SEQ ID NO: 4.

22. The cell of claim 17, wherein said genome comprises a functional disruption of both endogenous rhodopsin gene alleles.

23. The cell of claim 17, wherein said transgene is inserted into one or both endogenous rhodopsin gene alleles.

24. The cell of claim 17, which is an embryonic stem cell.

25. The cell of claim 17, which is in a mouse.

26. The cell of claim 17, which is isolated from a mouse.

27. The cell of claim 26, which is a rod cell.

28. An extract of the cell of claim 27, comprising an outer segment membrane of said cell.

29. A substantially purified transgenic polypeptide comprising a ROS targeting signal, isolated from the rod cell of claim 27, or from an extract thereof.

30. A mouse whose genome comprises:
a) a functional disruption of one or both endogenous rhodopsin gene alleles, and
b) a transgene encoding a polypeptide comprising a ROS targeting signal operably associated with a rod-specific regulatory sequence,
wherein said polypeptide is not a rhodopsin.

31. The mouse of claim 30, wherein said polypeptide is a GPCR.

32. The mouse of claim 31, wherein said GPCR is a cannabinoid receptor.

33. The mouse of claim 30, wherein said polypeptide is a fusion protein.

34. The mouse of claim 30, wherein said ROS targeting signal comprises SEQ ID NO: 4.

35. The mouse of claim 30, wherein said genome comprises a functional disruption of both endogenous rhodopsin gene alleles.

36. The mouse of claim 30, wherein said transgene is inserted into one or both endogenous rhodopsin gene alleles.

37. A rod cell or outer membrane extract thereof, isolated from the mouse of claim 30.

38. A substantially purified transgenic polypeptide comprising a ROS targeting signal, isolated from the rod cell or extract of claim 37.
